(19) United States
(12) Patent Application Publication
(10) Pub. No.: US 2021/0307732 A1
(43) Pub. Date: Oct. 7, 2021

(54) PORTABLE DRUG TESTING APPARATUS, SYSTEM, AND METHOD

(71) Applicant: BuzzKill Labs, Inc., Livermore, CA (US)

(72) Inventors: George FARQUAR, Livermore, CA (US); Jessica SEILHAN, Livermore, CA (US); Thomas O'LENIC, Livermore, CA (US); Marcus TOFANELLI, Livermore, CA (US)

(21) Appl. No.: 17/267,429

(22) PCT Filed: Aug. 12, 2019
(86) PCT No.: PCT/US19/46217

§ 371 Date: Feb. 9, 2021

Related U.S. Application Data
(60) Provisional application No. 62/718,851, filed on Aug. 14, 2018.

(51) Int. Cl. A61B 10/00; A61B 5/00; A61B 5/145

(52) U.S. Cl. CPC A61B 10/0051; A61B 5/4845; A61B 2560/0431; A61B 5/14507; A61B 2010/0009; A61B 5/4277

(57) ABSTRACT

A system for drug testing includes a collector configured to collect saliva, a processor configured to mate with the collector, and a detector. The collector comprises a mouthpiece, an elongate body, a vent to vent air from the body and a collection portion to collect saliva. The processor is configured to automatically extract a first sample of the saliva and a second sample of the saliva. The detector is configured to mate with the processor and to analyze the first sample to detect an amount of drug in the collected saliva.

### INCORPORATION BY REFERENCE

All publications and patent applications mentioned in this specification are herein incorporated by reference to the same extent as if each individual publication or patent application was specifically and individually indicated to be incorporated by reference.

### REFERENCE TO RELATED PATENT APPLICATIONS

This application claims the benefit of the filing date of U.S. Provisional Application 62/718,851, filed Aug. 14, 2018, the contents of which are incorporated herein by reference in their entirety for all purposes.

### BACKGROUND

Roadside driver tests to detect marijuana and other drugs are increasing in importance because drug availability and usage is increasing. Further, it is currently very difficult to enforce Driving Under the Influence of Drug (DUID) laws with existing oral fluid (i.e., saliva), breath, and blood tests. The lack of adequate roadside drug testing and effective DUID enforcement can result in a larger number of serious and/or fatal accidents.

According to an April 2017 poll, approximately 22% of American adults, or −54.5 million people, currently use marijuana, with 63% of this group indicating regular use. There are thus nearly as many marijuana users as there are cigarette smokers (˜59 million cigarette smokers). This number is expected to increase as marijuana legalization becomes more common. Additionally, the Department of Justice reports that the overall availability of controlled prescription and illegal drugs in the U.S. is also increasing or remaining stable at high rates. In a 2017 National Drug Threat report, the DEA noted that more individuals report current use of controlled prescription drugs than for cocaine, heroin, and methamphetamine combined, making controlled prescription drug use second only to marijuana.

As a result of increased drug usage, there are more drug-impaired drivers on the road than ever before. Indeed, drivers in fatal crashes are now more likely to be under the influence of drugs than alcohol. In Colorado, one of the first states to legalize recreational marijuana (2012), a November 2017 survey conducted by the Colorado Department of Transportation found that 55% of marijuana users believed it is safe to drive while under the influence. However, studies show that under the influence of THC, the psychoactive compound in marijuana, a user's reaction time and perception of distance and speed are both impaired.

There is currently not an efficient and reliable quantitative roadside test for marijuana and other drug use. FIG. 1 shows the current testing method, which requires that a Drug Recognition Expert (DRE) be called to the scene to perform a standard 12-step evaluation for impairment. Based upon the results of the DRE evaluation, the office can determine whether probable cause has been established and arrest the driver. Once arrested and transported to the station, blood tests can be performed and the results sent to the lab for evidentiary purposes. However, the use of Drug Recognition Experts (DREs) to establish probable cause is inefficient because: (a) it requires a trained expert, usually in addition to the detaining officer, doubling the manpower necessary, (b) it takes time for the expert to get to the scene once called, allowing drug levels to drop as drugs are metabolized, and (c) the DRE's subjective assessment still usually requires scientific evidence of drug presence through urinalysis or blood analysis by toxicologists to prosecute. Moreover, blood drawn hours later is not representative of the level of drugs present at the time of the traffic stop. Unlike alcohol, where the elimination rate is well understood, THC and other drug elimination rates are not consistent. Toxicologists therefore cannot perform retrograde extrapolation to estimate the amount of drugs present in a person's blood at a specific time in the past.

Accordingly, an efficient and reliable quantitative roadside test for marijuana and other drugs is desired.

### SUMMARY OF THE DISCLOSURE

In general, in one embodiment, a system for drug testing includes a collector configured to collect saliva, a processor configured to mate with the collector, and a detector. The processor is configured to automatically extract a first sample of the saliva and a second sample of the saliva. The detector is configured to mate with the processor and to analyze the first sample to detect an amount of drug in the collected saliva.

This and other embodiments can include one or more of the following features. The collector can be disposable. The processor can be disposable. The processor can be configured to fit within the detector. The system can be portable. The system can be less than 3,000 cubic inches. The system can weigh less than 10 kg. The processor can include automatic cleaning features therein for cleaning between uses. The processor can include a sample concentrator therein configured to automatically concentrate and prepare the first sample.

In general, in one embodiment, a device for collecting a saliva sample includes a hollow elongate body, a hollow bulb at a proximal end of the hollow elongate body, and a vent in fluid communication with the elongate body. The hollow bulb is configured to be chewed by the user. A proximal end of the hollow bulb has an opening therein to allow saliva to be transferred therethrough and into the hollow elongate body. The vent is configured to allow air to pass therethrough from the hollow elongate body.

In general, in one embodiment, a method of drug testing includes: (1) obtaining a saliva sample from a collector having a chewable bulb, (2) automatically generating a concentrated sample with a processor, and (3) inserting the concentrated sample into a portable detector to detect whether drug is present in the sample.

In one aspect, provided herein is a device for collecting a liquid sample, such as saliva, comprising: (a) a mouthpiece comprising an opening communicating with an internal space, the mouthpiece comprising a pliable material, and configured to receive the liquid, e.g., saliva, through the opening and into the space; (b) an elongate body comprising a lumen communicating with the space and configured to receive the liquid, e.g., saliva, from the internal space; (c) a vent, separate from the mouthpiece, comprising one or a plurality of apertures in fluid communication with the lumen and configured to allow air to pass therethrough and out of the device; and (d) a collection portion comprising a collection space configured to collect the liquid, e.g., saliva received into the lumen; wherein the device has dimensions configured to allow a user to chew the mouthpiece when the device inserted into the user's mouth. In one embodiment the device has a length between about 5 cm and about 20 cm, e.g., about 8 cm and about 15 cm. in another embodiment the mouthpiece has a bulb shape that is wider than the elongate body at a point of communication. In another embodiment the mouthpiece comprises a material selected from rubber and a plastic (e.g., low-density polyethylene, PVC, ABS, polystyrene, rubber, silicone, peek and tygon). In another embodiment the opening in the internal space has a volume of about 0.5 ml to about 5 ml, e.g., about 2 ml. In another embodiment the opening in the mouthpiece has a widest diameter of about 2 mm to about 10 mm, e.g., about 4 mm to about 8 mm. In another embodiment the mouthpiece comprises a composition that promotes salivation. In another embodiment the composition has a sour or sweet taste. In another embodiment the composition comprises citric acid, acetic acid malic acid, fumaric acid, and tartaric acid, sucrose, sodium chloride or an artificial sweetener (e.g., Splenda, saccharin, aspartame)). In another embodiment the lumen comprises surface features that promote breaking of bubbles in saliva. In another embodiment the vent comprises a plurality of apertures. In another embodiment the elongate body comprises a proximal portion positioned proximal to the mouthpiece and a distal portion positioned proximal to the collection portion, wherein the proximal portion is positioned radially within the distal portion wherein a plurality of the apertures are positioned radially with respect to an axis of the lumen. In another embodiment the proximal portion and the distal portion are configured as barrels wherein the proximal barrel is fitted radially into the distal barrel and defines an annular space between the two barrels, through which air can vent from the device. In another embodiment the elongate body comprises an elongate portion communicating through the one or more apertures with a vent tube oriented off-axis to the elongate member, wherein the vent tube comprises an aperture venting to air. In another embodiment the vent tube comprises a valve to inhibit escape of liquid from the vent tube. In another embodiment the one or a plurality of apertures are positioned between the mouthpiece and the collection space. In another embodiment the device is comprised in a single piece. In another embodiment the elongate body and the mouthpiece are comprised in separate pieces, fitted together. In another embodiment the elongate body and the mouthpiece are comprised in a single piece. In another embodiment the elongate body and the collection portion are comprised in separate pieces, fitted together. In another embodiment the elongate body and the collection portion are comprised in a single piece. In another embodiment the collection portion comprises a breakable seal which, when broken, allows collection of liquid from the collection portion. In another embodiment the seal is a friable seal. In another embodiment the seal comprises a foil. In another embodiment the device further comprises a mating element configured to mate the device with an interface of a processor and transmit liquid thereto. In another embodiment the mating element comprises a nipple, a tee, a valve, a screw lock or threaded tip (e.g., a luer lock) or a friction fit. In another embodiment the device further comprises a pump configured to promote air flow from the vent. In another embodiment the pump comprises a diaphragm pump. In another embodiment the pump exerts suction on the lumen.

In another aspect, provided herein is a method comprising: (a) providing a device comprising: (i) a mouthpiece comprising an opening communicating with an internal space, the mouthpiece comprising a pliable material, and configured to receive the liquid, e.g., saliva, through the opening and into the space; (ii) an elongate body comprising a lumen communicating with the space and configured to receive the liquid, e.g., saliva, from the internal space; (iii) a vent, separate from the mouthpiece, comprising one or a plurality of apertures in fluid communication with the lumen and configured to allow air to pass therethrough and out of the device; and (iv) a collection portion comprising a collection space configured to collect the liquid, e.g., saliva received into the lumen; wherein the device has dimensions configured to allow a user to chew the mouthpiece when the device inserted into the user's mouth; b) chewing on the mouthpiece of a device stimulate saliva production; c) collecting the saliva in collection space of the collection portion. In one embodiment the method further comprises using a pump to pump air out of the device. In another embodiment the method further comprises: d) detecting the presence of a drug in the collected liquid, e.g., saliva. In another embodiment the drug comprises a cannabinoid (e.g., delta-9-tetrahydrocannabinol (“THC”), cannabidiol (“CBD”), cannabinol (“CBN”), or tetrahydrocannabivarin (“THCV”). In another embodiment detecting is performed at the site of collecting (e.g., without sending the sample to laboratory remote from the point of collection. In another embodiment detecting is performed within 30 minutes of the time of collecting. In another embodiment collecting is performed by a law enforcement official. In another embodiment, collection is performed at a roadside. In another embodiment, collection can be performed at a location remote from detection, for example, in another building, in another city, at least 1 mile from the point of collection, at least 5 miles from the point of collection or at least 25 miles from the point collection.

In another aspect provided herein is a system for drug testing, comprising: a collector configured to collect saliva; a processor configured to mate with the collector, the processor configured to automatically extract a first sample of the saliva and a second sample of the saliva; and a detector configured to mate with the processor, the detector configured to analyze the first sample to detect an amount of drug in the collected saliva. In one embodiment the collector is disposable. In another embodiment the processor is disposable. In another embodiment the processor is configured to fit within the detector. In another embodiment the system is portable. In another embodiment the system is less than 3,000 cubic inches. In another embodiment the system weighs less than 10 kg. In another embodiment the processor includes automatic cleaning features therein for cleaning between uses. In another embodiment the processor includes a sample concentrator therein configured to automatically concentrate and prepare the first sample.

In another aspect provided herein is a device for collecting a saliva sample, comprising: a hollow elongate body; hollow bulb at a proximal end of the hollow elongate body configured to be chewed by the user, a proximal end of the hollow bulb having an opening therein to allow saliva to be transferred therethrough and into the hollow elongate body; and a vent in fluid communication with the hollow elongate body and configured to allow air to pass therethrough from the hollow elongate body.

In another aspect provided herein is a method of drug testing comprising: obtaining a saliva sample from a collector having a chewable bulb; automatically generating a concentrated sample with a processor; and inserting the concentrated sample into a portable detector to detect whether drug is present in the sample.

### BRIEF DESCRIPTION OF THE DRAWINGS

The novel features of the invention are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIG. 1 shows a prior method of roadside drug testing.

FIG. 2 shows a method of roadside drug testing as described herein.

FIGS. 3A and 3B show a drug testing apparatus.

FIG. 4A shows a saliva collector.

FIGS. 4B-4C show a saliva collector with an off-axis vent.

FIGS. 5A-5B show a collector and fluidic processor.

FIG. 5C is a diagram of a fluidic processor.

FIG. 6 shows a sample extraction process of THC and THCA.

FIG. 7 shows a quantitative display for a drug testing apparatus.

FIG. 8 shows a portable drug testing apparatus.

FIG. 9 is a flow chart for drug testing as described herein.

FIG. 10 shows an embodiment of a device comprising vents.

FIG. 11 shows a device comprising a pump configured to apply suction to an internal space in an elongate body configured to collect liquid.

### DETAILED DESCRIPTION

Described herein are portable drug testing apparatuses, systems, and methods that can be efficiently and reliably used to both collect evidentiary oral fluid samples and collect and analyze oral fluid samples to provide quantitative results quickly for driver impairing substances. The apparatuses, systems, and methods described herein can be performed on-site (e.g., on the roadside), can be fully automated, and can provide quantitative results for a large number of substances.

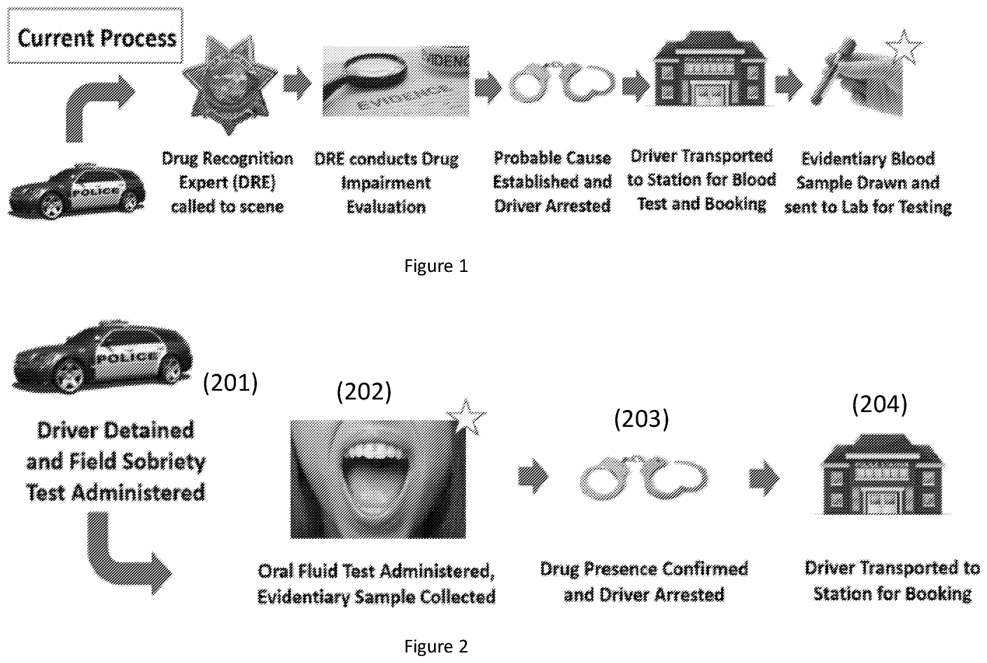

Referring to FIG. 2, a method for testing for impairing substances can include, at step 201, detaining the driver. At step 202, an oral fluid test can be administered, including gathering an evidentiary sample. At step 203, the presence of the impairing substance can be confirmed and, if necessary, the driver arrested. At step 204, the driver can be transported to the station for booking. Such a method can thus greatly reduce the number of steps required to evaluation impairment relative to the method shown in FIG. 1.

### I. System

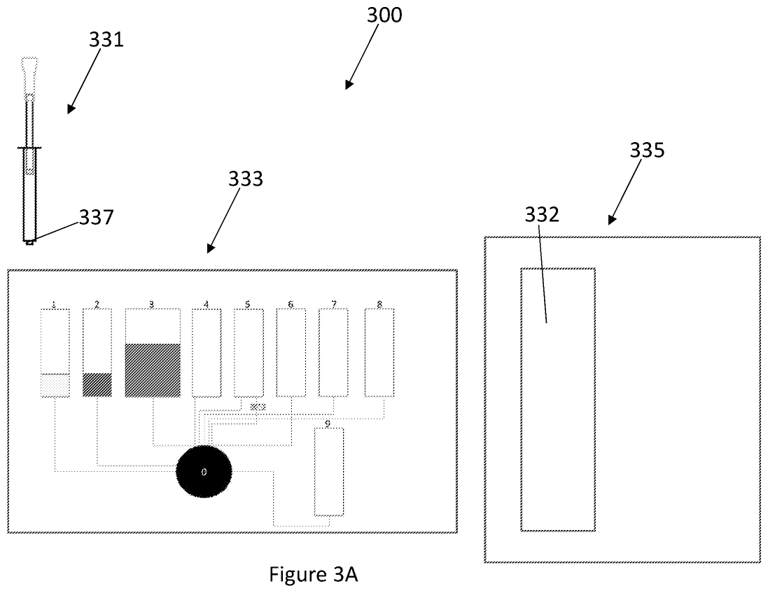

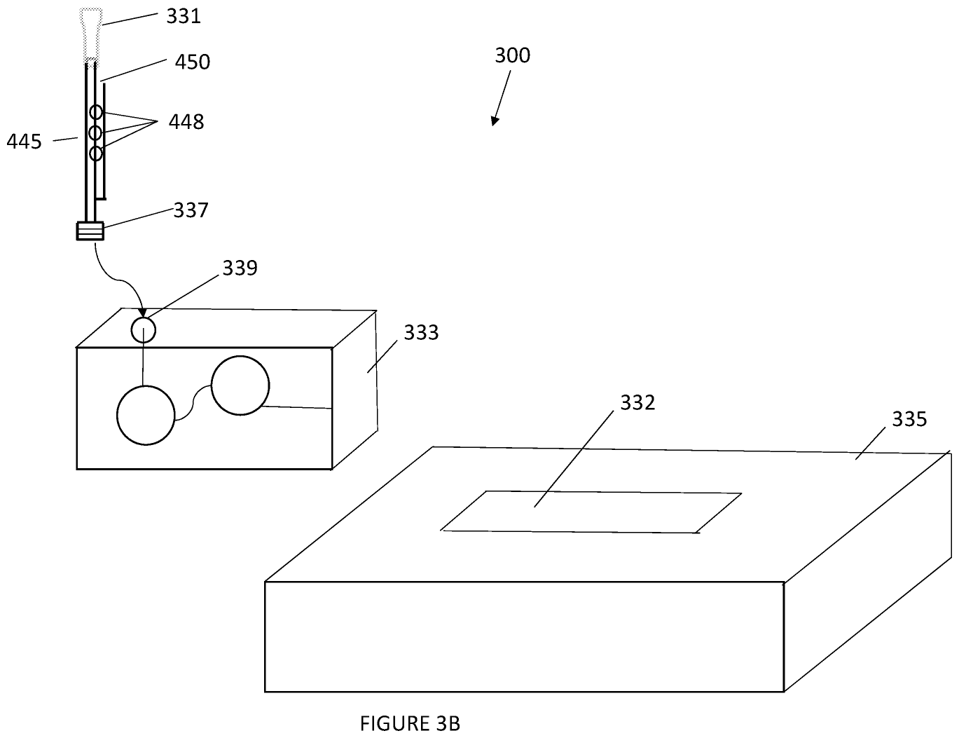

An exemplary system 300 for testing for impairing substances is shown in FIGS. 3A and 3B. The system 300 includes a collector device 331, a fluidic processor 333, and a detector 335. The collector device 331 can be configured to collect oral fluid (e.g., saliva) from the user, e.g., a driver. Further, the collector device 331 can be configured to mate with the fluidic processor 333 to transfer the collected fluid or liquid thereto. The collector 331 can include a mating element. For example, the mating element 337 can comprise a threaded tip that is configured to mate with a threaded opening 339 in the fluidic processor 333. In addition, the collector device 331 can be fitted with a breakable seal at the distal end thereof, e.g., a friable seal. The seal can be pierced when connected to the processor 333, for example, with a pin or pointed object positioned in the fluidic processor interface. The fluidic processor 333, in turn, can be configured to process the collected fluid (e.g., extract the sample and prepare a secondary sample). The fluidic processor 333 can mate with the detector 335 to transfer the extracted sample thereto for analysis. For example, the entire fluidic processor 333 can fit within a slot 332 in the detector 335. The fluidic processor 333 can be disposable or reusable. When reusable, the processor 333 can include automatic cleaning features therein configured to clean the chambers and elements therein between uses. In some embodiments, the fluidic processor 333 can be permanently attached to the detector 335. When permanently attached, the fluidic processor 333 and detector 335 can have additional cleaning options for reuse between samples.

### II. Collection Device

A saliva collection device of this disclosure includes: (a) a mouthpiece comprising an opening communicating with an internal space, the mouthpiece comprising a pliable material, and configured to receive saliva through the opening and into the space; (b) an elongate body comprising a lumen communicating with the space and configured to receive saliva from the internal space; (c) a vent, separate from the mouthpiece, comprising one or a plurality of apertures in fluid communication with the lumen and configured to allow air to pass therethrough and out of the device; and (d) a collection portion comprising a collection space configured to collect fluid received into the lumen; wherein the device has dimensions configured to allow a user to chew the mouthpiece when the device inserted into the user's mouth.

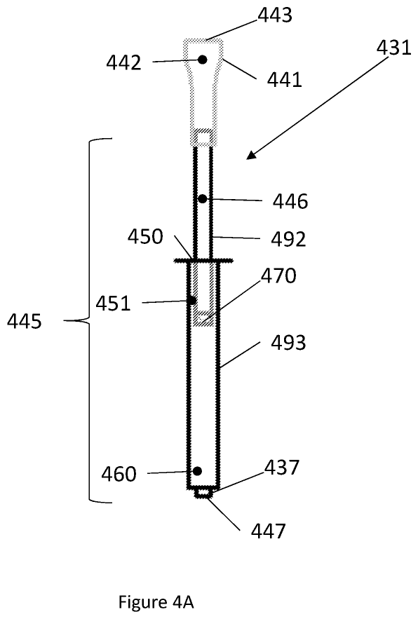

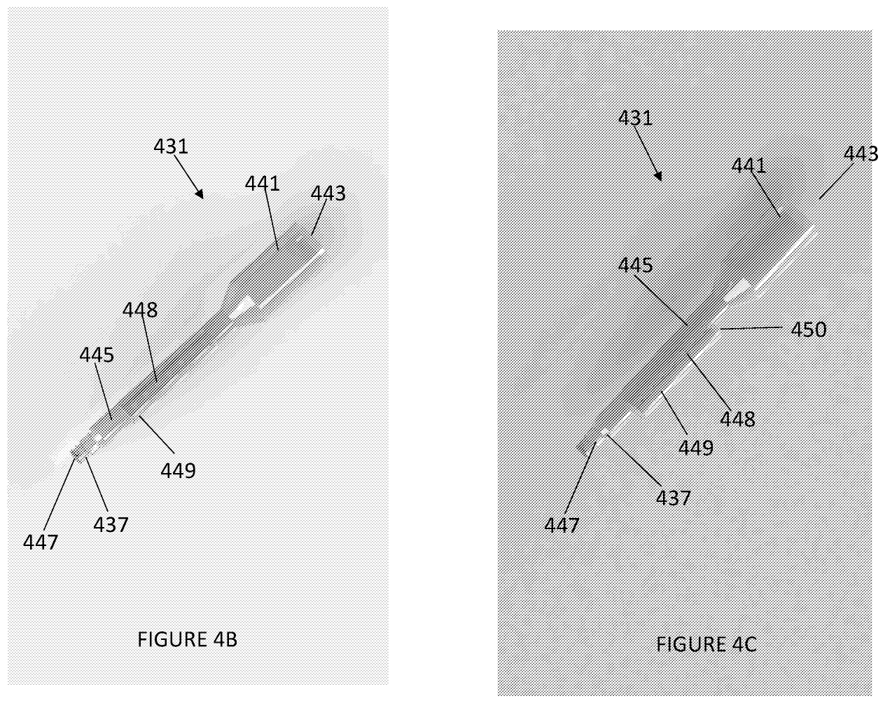

A close-up of an exemplary collector device 431 is shown in FIG. 4A. The collector 431 can include a hollow mouthpiece 441 having an internal space 442 and being fluidically connected to an elongate member 445 having a lumen 446. The mouthpiece 441 can be made of a relatively pliable material, such as rubber or a plastic (e.g., low-density polyethylene, PVC, ABS, polystyrene, rubber, silicone, peek, tygon) having a chewable texture. The mouthpiece can take a bulb shape, that is, comprising a portion wider than the elongate member to which it is attached, e.g., through a stem. So, for example, the mouthpiece can be formed from the bulb of an eye dropper or the like. The mouthpiece can include a proximal opening 443 therein configured to receive liquids, such as saliva, from the mouth of a user into the internal space 442. The internal space can have a volume of about 0.5 mL to about 5 mL, e.g., about 2 mL.

Further, the device 431 can comprise a collection portion at an end of the elongate member distal from the mouthpiece and comprising a collection space 460 to collect liquid received from the mouthpiece into the lumen.

The elongate member can terminate distal to the mouthpiece in a mating element 437 configured to mate with an interface of a processor. The mating element 437 can comprise, for example, a nipple, a tee, a valve, a screw lock or a threaded tip (e.g., a luer lock), or a friction fit. The mating element 437 typically includes an opening that is typically sealed with a removable or breakable seal 447. So, for example, the mating element 437 can comprise a threaded tip. The sealed opening 447 (e.g., a plastic or foil seal) can retain the oral fluid sample until the collector device 431 is mated with the processor. Upon mating, the seal can break. This can be a college, for example, by providing a piercing or tearing element such as a pin or a sharp protrusion. In some embodiments, the elongate member 445 can include markers thereon to indicate the volume of collected fluid. In some embodiments the elongate member can have antioxidants to help preserve the sample. In some embodiments the elongate member can be coated with pH buffering material. The buffer can be dry or liquid. The pH buffer aids in stabilizing and preparing the oral fluid for analysis. In some embodiments the elongate member 445 can be curved to allow for collection of oral fluid near the bottom of the mouth. In some embodiments, the elongate member 445 can include a sensor to indicate the volume of collected fluid.

In use, the user (e.g., the driver or test subject) can place the mouthpiece 441 into the mouth with the elongate member 445 positioned substantially vertically (e.g., with the distal end 437 pointed downwards). The user can then chew on the mouthpiece 441 to activate the salivary glands. Accordingly, the device 431 can have dimensions such that when inserted into the mouth, the mouthpiece can be chewed, for example, by the molars of an adult human user. The saliva can drop downwards through the opening 443 into space 442 of the mouthpiece 441 and onwards into the lumen 446 of hollow elongate member 445. Advantageously, the salivary glands can be activated by chewing on the mouthpiece 441 even if the user is experiencing dry mouth, e.g., caused by tetrahydrocannabinol (THC). The user can continue to chew on the mouthpiece 441 until the desired amount of saliva has been collected in the hollow elongate member 445. In some embodiments, the mouthpiece 441 can comprise, e.g., be coated (externally or internally) with a chemical composition to enhance oral fluid production (e.g., citric acid, acetic acid malic acid, fumaric acid, and tartaric acid, sucrose, sodium chloride, and artificial sweetener (e.g., Splenda, saccharin, aspartame)).

Samples collected by the device may be frothy, that is, may contain bubbles. These bubbles may interfere with collection of liquid in the sample. Accordingly, device 431 can comprise a venting mechanism such that gas released from bubbles is vented from the device into the environment, e.g., the air. The venting mechanism can comprise one or a plurality of apertures in the device to allow passage of gas from the lumen of the elongate body into the environment.

Collection devices of this disclosure comprise a vent to allow air to escape from spaces internal to the device. The vent includes one or more apertures in the elongate member that communicate with the environment, e.g., with air.

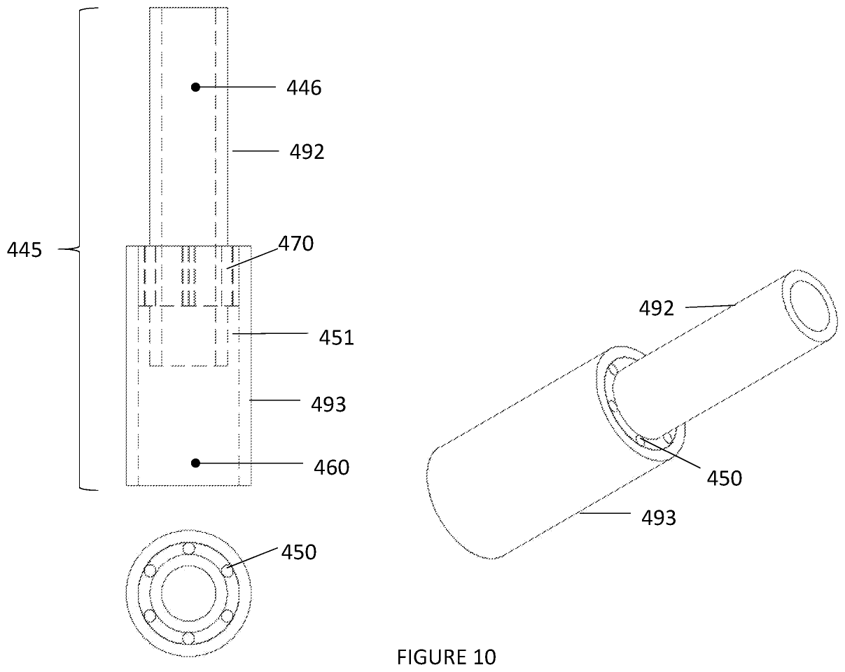

One embodiment of a vent is shown in FIG. 4 and FIG. 10. Elongate member 445 comprises proximal portion 492 positioned radially within larger diameter distal portion 493. In this embodiment, proximal portion 492 comprises flared portion 470 that comprises a plurality of vent holes (apertures) 450 that vent to air. The proximal and distal portions of the member can be configured as barrels, with diameters such that a space 451, e.g., an annular space, exists between an outer wall of the first barrel and an inner wall of the second barrel. In this embodiment, saliva flows down through lumen 446 to collection space 460 causing bubbles to burst. The elongate member can comprise features, e.g., surface features, to promote breaking of bubbles. Such features can include, for example, roughness or bumps on surfaces that come into contact with saliva or sharp edges near or at the apertures. Air flows out of the bottom of proximal portion 492, around distal portion 493, into annular space 451 and out of the vent holes 450. This allows the air and oral fluid to be radially separated.

In some embodiments the collection portion 493 is split into two collectors. This allows one sample to be archived for later analysis.

In another embodiment, as shown in FIGS. 4B and 4C, the vent tube portion 449 (with holes 448 into a lumen of the elongate member) can extend off-axis relative to the elongate tube 445, which can prevent oral fluid from entering the vent tube 449. Gas can exit the device through a vent hole 450 in the vent tube. A liquid prevention or one-way valve can alternatively or additionally be added to the vent tube 449 to prevent the escape of oral fluid. The vent one-way valve 453 can be made of a flap valve a spring-loaded ball bearing valve or other valve preventing the liquid from escaping. The vent tube 449 can advantageously vent air from the elongate tube 445 to prevent bubbling of collected saliva within the elongate tube 445.

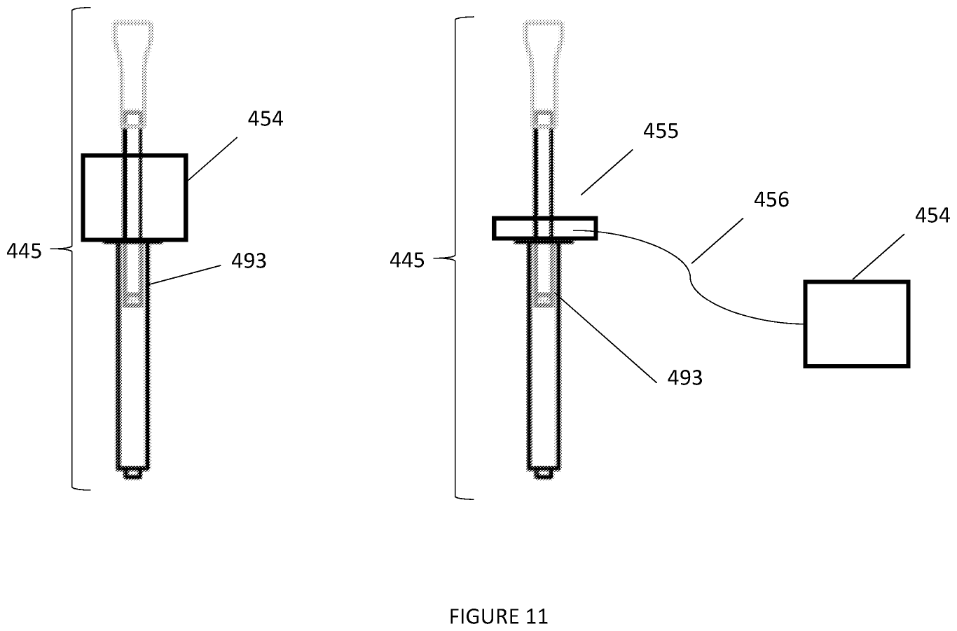

The vent can have a pump attached. The pump removes the air and assists in collecting the oral fluid. The pump can be battery operated or manual operated. The pump can be disposable or reusable. The pump can comprise a diaphragm pump, a syringe pump or other pump. FIG. 11 shows the device 445 with the pump 454 attached. The pump enhances the removal of the air and assists in collecting of the oral fluid by creating a lower pressure, e.g., a source of vacuum, in the device 445. In some embodiments the pump 454 can be attached with a connector 455 and tube 456 to the device 445.

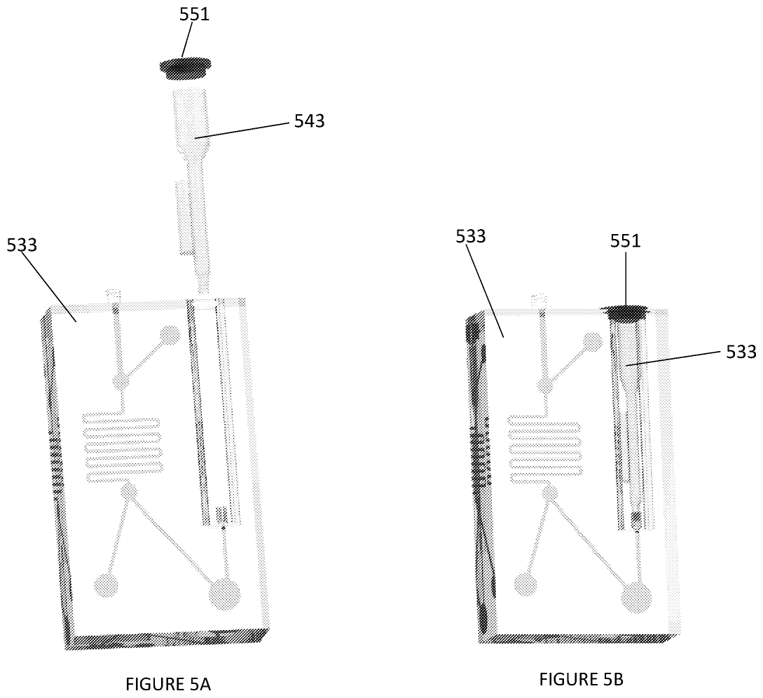

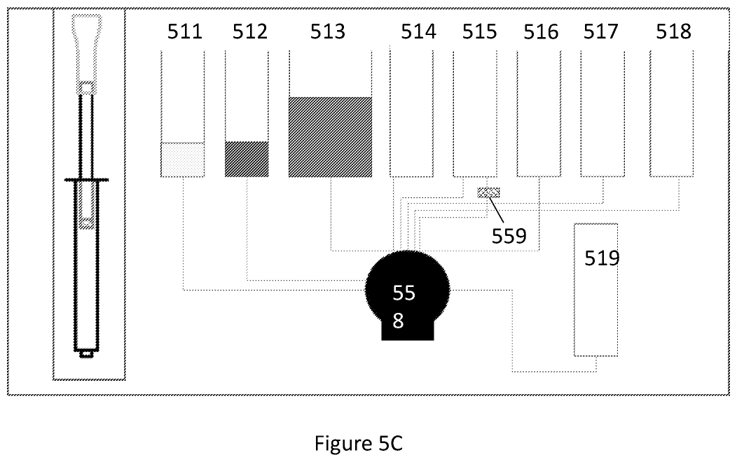

Referring to FIGS. 5A-5B, the collector 531 can have a proximal cap 551 configured to fit over the proximal opening 543 to prevent spillage of the collected oral fluid during transfer. The collector 531 can be configured to sit within a slot 551 in the fluidic processor 533 and to transfer fluid thereto.

### III. Processor

As shown in FIG. 5C, the processor 533 can include a collection chamber 511, a buffer chamber 512, a solvent chamber 513, a mixing chamber 514, an emulsion breaking chamber 515, an extract chamber 516, a final collection chamber 517 for the processed sample, an evidentiary sample chamber 518, and a pump 519 (e.g., a syringe pump) to move fluid through the processor 533. The chambers 511-518 and pump 519 can be fluidically connected through fluid lines and a valve 558 (e.g., a 12-way valve). A filter 559 can be fluidically connected to the emulsion breaking chamber 515. In some embodiments, the processor 533 can include one or more additional reagent chambers (e.g., a chemical modification reagent chamber).

THC and other drugs are known to degrade over time due to oxygen. For samples that require prolonged storage a radical/oxygen reagent can be added, such as Butylated hydroxytoluene or other known preservatives. In addition, a secondary compound which has a decay that correlates with THC can be added in order to determine the amount of THC at time of collection. This includes but is not limited to synthetic cannabinoids (not found in nature).

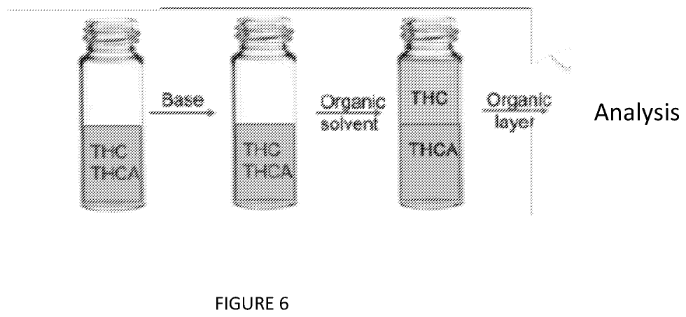

The processor 533 can be used to extract the chemical(s) for which testing is being performed from the saliva sample. For example, the processor 533 can be configured to extract THC from the sample. Such extraction can be useful because tetrahydrocannabinolic acid (THCA), the carboxylic acid form of THC, is present in marijuana in high concentrations, sometimes exceeding the THC concentration. THCA is not psychoactive, and the presence in oral fluid is not an indicator of intoxication. However, THCA can cause false positives in analysis. FIG. 6 shows the sample extraction process and partitioning of THC and THCA into two phases using a base and organic solvent (e.g., hexane: ethyl acetate, octane: ethyl acetate, pentane: ethyl acetate, or neat ethyl acetate with NaCl). The organic top layer, including the THC, can then be used for analysis. In some embodiments, the extracted sample is enhanced 10X relative to the gathered sample. For example, lml can be gathered and a 100 μl extracted sample can be produced. The processor 533 can be fully automated to perform the extraction and sample preparation.

Additionally, in some embodiments, the processor 533 can be configured to separate the extracted sample into separate samples—one for analysis and one for storage (e.g., for evidentiary purposes and/or further analysis). The second sample (e.g., for evidentiary purposes) can be configured to be stored within the processor 533 until it is needed. The two samples can each be, for example, between 1μ-10 ml in size.

Referring back to FIG. 3A-3B, the processor (333 or 533) can be configured to be placed within a portable detector (e.g., detector 335). The detector 335 can obtain the extracted sample through a sample handling line fluidically connecting the processor 333 to the detector 335 and can be configured to analyze for the presence of impairing substances. The detector can use, for example, a separation technique including column chromatography, ion-exchange chromatography, gel-permeation (molecular sieve) chromatography, affinity chromatography, gas chromatography, paper chromatography, thin-layer chromatography, gas chromatography, dye-ligand chromatography, hydrophobic interaction chromatography, pseudoaffinity chromatography, high-pressure liquid chromatography (HPLC), ion mobility, vacuum chromatography, or flash chromatography. Further, the detector can use a detection technique including an ion mobility detector (IM), a charged aerosol detector (CAD), a flame ionization detector (FID), an aerosol-based detector (NQA), a flame photometric detector (FPD), an atomic-emission detector (AED), a nitrogen phosphorus detector (NPD), a photo ionization detector (PID), an evaporative light scattering detector (ELSD), a mass spectrometer (MS), an electrolytic conductivity detector (ELCD), a sumon detector (SMSD), a mira detector (MD), an ultraviolet (UV) detector, a thermal conductivity detector, a fluorescence detector, an electron capture detector (ECD), a conductivity monitor, a refractive index detector (RI or RID), a radio flow detector, a chiral detector, a microelectromechanical (MEMS) detector, a cantilever detector, a photomultiplier tube (PMT) detector, a mass selective detector, a micro channel plate detector, a Dielectric Barrier Detector (PBD), or an electron multiplier detector. The results of the detection mechanism can be quantitative and can be accurate at concentrations of 0.001 ng/ml and greater, such as 0.01 ng/ml and greater. The detection limit can thus be below 0.01 ng/ml. In some embodiments, the detector can include a concentrator device that will allow the analyte to be concentrated to a small volume. For example, the sample may be heated to remove any solvent and concentrate the analyte. Other techniques of sample concentration include solid phase micro extraction (SPME), solid phase extraction (SPE), sorption and ion exchange.

### IV. Detector

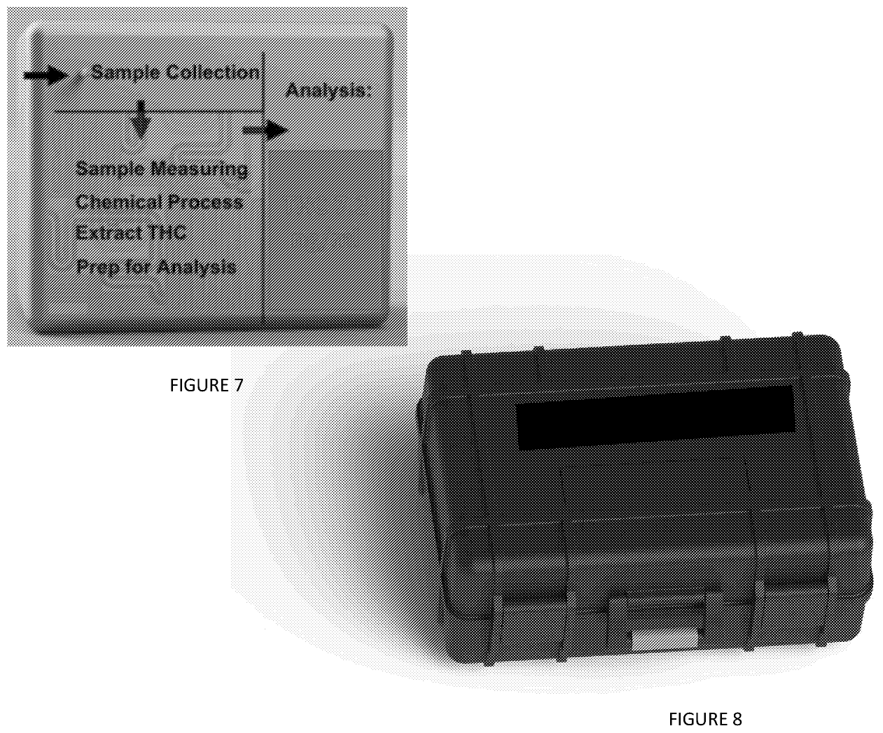

In some embodiments, as shown in FIG. 7, the results from the analysis can be displayed in a quantitative display on the detector or device. Such quantitation can be obtained through methods such as internal standards, calibration data, and total ion signal. In some embodiments, the sample can be mass selected, and an internal standard of the analyte can be isotopically labeled. Additionally, in some embodiments, the results can be sent to a phone and/or remote computer or cloud for further analysis or storage.

As shown in FIG. 8, the detector (and the attached processor) can be configured to fit within a case, e.g., for placement in the trunk of a police car. The overall device, therefore can be less than 3,000 cubic inches, such as less than 2,500 cubic inches in volume. In some embodiments, the detector can use less than 300 watts of power. Further, the detector (and entire device) can be lightweight, e.g., weigh less than 10 kg.

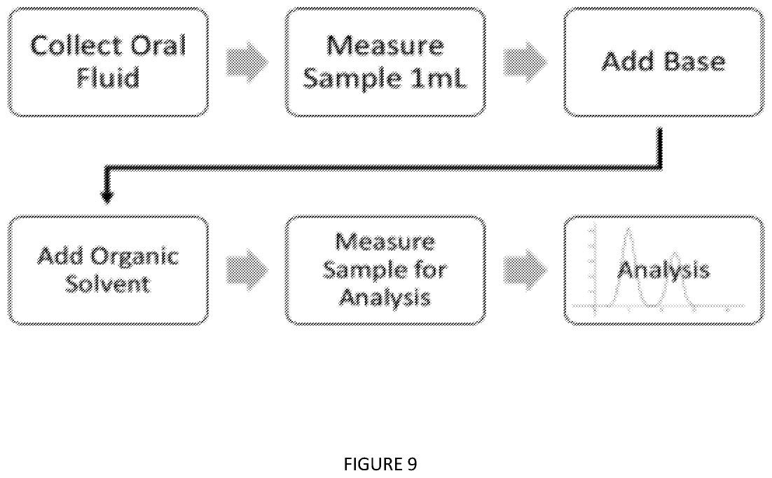

The overall method of gathering the oral fluid through analysis is shown in FIG. 9. In some embodiments, the steps that occur after collection through analysis can take 10 minutes or less. The devices, systems, and methods described herein can thus advantageously result in a quantitative test for impairing substances (e.g., THC) directly at the location (e.g., roadside) in less than 15 minutes from the start of the analysis (e.g., the user chewing on the bulb). Additionally, a sample can be safely and efficiently stored for later evidentiary use.

It should be understood that any feature described herein with respect to one embodiment can be substituted for, or used in addition to, any feature described herein with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps. The term “consisting essentially of” refers to the inclusion of recited elements and other elements that do not materially affect the basic and novel characteristics of a claimed combination.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A device for collecting a saliva sample, comprising:
(a) a mouthpiece comprising an opening communicating with an internal space, the mouthpiece comprising a pliable material, and configured to receive saliva through the opening and into the space;
(b) an elongate body comprising a lumen communicating with the space and configured to receive saliva from the internal space;
(c) a vent, separate from the mouthpiece, comprising one or a plurality of apertures in fluid communication with the lumen and configured to allow air to pass therethrough and out of the device; and
(d) a collection portion comprising a collection space configured to collect fluid received into the lumen;
wherein the device has dimensions configured to allow a user to chew the mouthpiece when the device inserted into the user's mouth.

2. The device of claim 1, having a length between about 5 cm and about 20 cm, e.g., about 8 cm and about 15 cm.

3. The device of claim 1, wherein the mouthpiece has a bulb shape that is wider than the elongate body at a point of communication.

4. The device of claim 1, wherein the mouthpiece comprises a material selected from rubber and a plastic (e.g., low-density polyethylene, PVC, ABS, polystyrene, rubber, silicone, peek and tygon).

5. The device of claim 1, wherein the opening in the internal space has a volume of about 0.5 ml to about 5 ml, e.g., about 2 ml.

6. The device of claim 1, wherein the opening in the mouthpiece has a widest diameter of about 2 mm to about 10 mm, e.g., about 4 mm to about 8 mm.

7. The device of claim 1, wherein the mouthpiece comprises a composition that promotes salivation.

8. The device of claim 7, wherein the composition has a sour or sweet taste.

9. The device of claim 7, wherein the composition comprises citric acid, acetic acid malic acid, fumaric acid, and tartaric acid, sucrose, sodium chloride or an artificial sweetener (e.g., Splenda, saccharin, aspartame)).

10. The device of claim 1, wherein the lumen comprises surface features that promote breaking of bubbles in saliva.

11. The device of claim 1, wherein the vent comprises a plurality of apertures.

12. The device of claim 11, wherein the elongate body comprises a proximal portion positioned proximal to the mouthpiece and a distal portion positioned proximal to the collection portion, wherein the proximal portion is positioned radially within the distal portion wherein a plurality of the apertures are positioned radially with respect to an axis of the lumen.

13. The device of claim 12, wherein the proximal portion and the distal portion are configured as barrels wherein the proximal barrel is fitted radially into the distal barrel and defines an annular space between the two barrels, through which air can vent from the device.

14. The device of claim 11, wherein the elongate body comprises an elongate portion communicating through the one or more apertures with a vent tube oriented off-axis to the elongate member, wherein the vent tube comprises an aperture venting to air.

15. The device of claim 14, wherein the vent tube comprises a valve to inhibit escape of liquid from the vent tube.

16. The device of claim 1, wherein the one or a plurality of apertures are positioned between the mouthpiece and the collection space.

17. The device of claim 1, wherein the device is comprised in a single piece.

18. The device of claim 1, wherein the elongate body and the mouthpiece are comprised in separate pieces, fitted together.

19. The device of claim 1, wherein the elongate body and the mouthpiece are comprised in a single piece.

20. The device of claim 1, wherein the elongate body and the collection portion are comprised in separate pieces, fitted together.

21. The device of claim 1, wherein the elongate body and the collection portion are comprised in a single piece.

22. The device of claim 1, wherein the collection portion comprises a breakable seal which, when broken, allows collection of liquid from the collection portion.

23. The device of claim 22, wherein the seal is a friable seal.

24. The device of claim 22, wherein the seal comprises a foil.

25. The device of claim 1, further comprising a mating element configured to mate the device with an interface of a processor and transmit liquid thereto.

26. The device of claim 25, wherein the mating element comprises a nipple, a tee, a valve, a screw lock or threaded tip (e.g., a luer lock) or a friction fit.

27. The device of claim 1, further comprising a pump configured to promote air flow from the vent.

28. The device of claim 27, wherein the pump comprises a diaphragm pump.

29. The device of claim 27, wherein the pump exerts suction on the lumen.

30. A method comprising:
(a) providing a device comprising:
(i) a mouthpiece comprising an opening communicating with an internal space, the mouthpiece comprising a pliable material, and configured to receive saliva through the opening and into the space;
(ii) an elongate body comprising a lumen communicating with the space and configured to receive saliva from the internal space;
(iii) a vent, separate from the mouthpiece, comprising one or a plurality of apertures in fluid communication with the lumen and configured to allow air to pass therethrough and out of the device; and
(iv) a collection portion comprising a collection space configured to collect fluid received into the lumen;
wherein the device has dimensions configured to allow a user to chew the mouthpiece when the device inserted into the user's mouth;
b) chewing on the mouthpiece of a device stimulate saliva production;
c) collecting the saliva in collection space of the collection portion.

31. The method of claim 30, further comprising using a pump to pump air out of the device.

32. The method of claim 30, further comprising:
d) detecting the presence of a drug in the collected saliva.

33. The method of claim 32, wherein the drug comprises a cannabinoid (e.g., delta-9-tetrahydrocannabinol (“THC”), cannabidiol (“CBD”), cannabinol (“CBN”), or tetrahydrocannabivarin (“THCV”).

34. The method of claim 32, wherein detecting is performed at the site of collecting (e.g., without sending the sample to laboratory remote from the point of collection.

35. The method of claim 32, wherein detecting is performed within 30 minutes of the time of collecting.

36. A system for drug testing, comprising:
a collector configured to collect saliva;
a processor configured to mate with the collector, the processor configured to automatically extract a first sample of the saliva and a second sample of the saliva; and
a detector configured to mate with the processor, the detector configured to analyze the first sample to detect an amount of drug in the collected saliva.

37. The system of claim 34, wherein the collector is disposable.

38. The system of claim 34, wherein the processor is disposable.

39. The system of claim 34, wherein the processor is configured to fit within the detector.

40. The system of claim 34, wherein the system is portable.

41. The system of claim 34, wherein the system is less than 3,000 cubic inches.

42. The system of claim 34, wherein the system weighs less than 10 kg.

43. The system of claim 34, wherein the processor includes automatic cleaning features therein for cleaning between uses.

44. The system of claim 34, wherein the processor includes a sample concentrator therein configured to automatically concentrate and prepare the first sample.

45. A device for collecting a saliva sample, comprising:
a hollow elongate body;
hollow bulb at a proximal end of the hollow elongate body configured to be chewed by the user, a proximal end of the hollow bulb having an opening therein to allow saliva to be transferred therethrough and into the hollow elongate body; and
a vent in fluid communication with the hollow elongate body and configured to allow air to pass therethrough from the hollow elongate body.

46. A method of drug testing comprising:
obtaining a saliva sample from a collector having a chewable bulb;
automatically generating a concentrated sample with a processor; and
inserting the concentrated sample into a portable detector to detect whether drug is present in the sample.
